(19) United States
(12) Patent Application Publication
(10) Pub. No.: US 2023/0166991 A1
(43) Pub. Date: Jun. 1, 2023

(54) Methods and systems for streaming current analyzer calibration and reporting

(71) Applicant: Marmac Water LLC, Greenwood Village, CO (US)

(72) Inventors: Mary Teresa Marvelli, Greenwood Village, CO (US); Gregg Allan McLeod, Greenwood Village, CO (US)

(21) Appl. No.: 17/994,890

(22) Filed: Nov. 28, 2022

Related U.S. Application Data
(60) Provisional application No. 63/361,081, filed on Nov. 26, 2021.

(51) Int. Cl. C02F 1/52; G01N 33/18; B01L 3/00

(52) U.S. Cl. CPC C02F 1/5209; G01N 33/18; C02F 1/5245; B01L 3/502715; C02F 2209/11; C02F 2209/003; C02F 2209/006; C02F 2209/05; C02F 2209/10; C02F 2209/07; C02F 2209/02; C02F 2209/06; C02F 2103/001

(57) ABSTRACT

Disclosed are methods and systems that facilitate more accurate measurement of the turbidity, i.e., insoluble ion content, of source water streams in water treatment processes, and in turn more accurate dosing of metal salt coagulants to cause these insoluble ions to floc and precipitate from the source water stream. Methods and systems for calibration of streaming current sensors used for such turbidity/insoluble ion content measurement are also disclosed.

### CROSS REFERENCE TO RELATED APPLICATION

This application claims the benefit of priority under 35 U.S.C. § 119(e) of U.S. Provisional Patent Application 63/361,081, filed 26 Nov. 2021, the entirety of which is incorporated herein by reference.

### FIELD

This disclosure relates generally to streaming current analyzers used in water treatment and similar applications, and particularly to methods for automatically calibrating and improving the accuracy of such analyzers.

### BACKGROUND

Metal salt coagulants, such as aluminum sulfate, aluminum chlorhydrate, polyaluminum chloride, ferric chloride, and ferric sulfate, are used in water treatment processes to remove solid particulates from the source water via charge neutralization and thereby reduce the turbidity of the water. Particularly, the particles that cause turbidity generally possess an anionic charge and are therefore attracted to cationic coagulant particles; the resulting ionic compounds then precipitate out of the source water.

As those skilled in the art appreciate, the turbidity level of the source water in many water treatment processes (e.g., municipal wastewater treatment) may be subject to frequent, rapid, and/or unexpected variations, due to, e.g., time of day, time of year, recent weather, etc. When the turbidity level of the source water changes, so too does the coagulant dosing required to neutralize the anionic charge of the particles in the water and cause them to precipitate. The optimal coagulant dose is one that exactly offsets the anionic charge of the particles, such that the source water has a net electric charge (i.e., net zeta potential) of zero. Thus, a typical wastewater treatment process or system, especially one in which at least some degree of automation of coagulant dosing is desired, will utilize zeta potential and/or streaming current analyzers to assess changes in turbidity levels (and thus coagulant dose demand) in real time. Like almost all measuring tools, however, streaming current analyzers can, over time, suffer from “drift,” i.e., reduced accuracy and increased measurement errors, which may be due to any of several factors (e.g., sudden shocks, environmental changes, vibration, normal wear and tear, debris buildup, electromagnetic interference, and the like); this measurement drift has limited the market penetration of automated coagulant dosing, and in most systems the streaming current readings are used merely as a guide to allow operators to adjust the coagulant dose manually.

Most currently existing streaming current analysis and monitoring systems, such as those manufactured and/or sold by companies such as Chemtrac and Milton Roy, have multiple different settings for the sensitivity, or “gain,” of the sensor. In the most sensitive (i.e., smallest-gain) settings, these analyzers can recognize minute changes in the turbidity (i.e., non-dissolved particle content) of the source water, but will also detect soluble ions, such as ions that cause alkalinity/water hardness (e.g., calcium ions, magnesium ions) and organic ions. Because soluble ions are typically treated/removed using methods other than metal salt coagulant dosing, detection of these ions by the streaming current analyzer can reduce the effectiveness of any automated coagulant dosing scheme and can obfuscate changes in turbidity if the soluble ion content simultaneously changes in the opposite direction (e.g., after storm events, which typically decrease soluble ion content but increase turbidity). On the other hand, the use of a larger-gain (i.e., less sensitive) setting can prevent the streaming current analyzer from detecting soluble ions but will also necessarily reduce the accuracy of turbidity measurements and the sensitivity of the system to changes in turbidity. Thus, presently available streaming current analyzers and/or monitors are especially ineffective when used to assess the zeta potential of coagulant floc formation in highly alkaline source water streams, i.e., source water that is highly resistant to acidification and thus tends to maintain a stable pH.

There is thus a need in the art for methods and systems for determining the streaming current of a source water stream that reduce or eliminate measurement drift, and/or that are less prone to detecting soluble ions while remaining highly sensitive to changes in water turbidity.

### SUMMARY

The present disclosure provides methods and systems for calibration of streaming current analyzers, detectors, monitors, and/or sensors in water treatment processes and systems. Particularly, the methods and systems of the present disclosure can correct for inaccuracies in streaming current measurement caused by highly alkaline source water streams by determining the true alkalinity of the water and, based upon the true alkalinity reading, placing upper and lower limits on the readings of streaming current measurement equipment and/or on the amount of coagulant dispensed by an automated coagulant dosing system. In this way, the methods and systems of the present disclosure can effectively compensate for exaggerated or erroneous measurements of zeta potential and/or streaming current, particularly in highly alkaline source water streams.

In one aspect of the present disclosure, a method for monitoring the streaming current of a source water stream in a water treatment process comprises (a) calculating, based on data collected by first and second ion content sensors, a soluble ion content-compensated turbidity of the source water stream at or upstream of a coagulant dosing device, wherein the first ion content sensor is positioned at or upstream of the coagulant dosing device and is configured to collect data relating to a combined content of insoluble and soluble ions in the source water stream, and wherein the second ion content sensor is positioned downstream of the coagulant dosing device and is configured to collect data relating to a content of soluble ions in the source water stream; and (b) at least one of (i) displaying the soluble ion content-compensated turbidity of the source water stream in a graphical user interface of a computer and (ii) communicating the soluble ion content-compensated turbidity of the source water stream to a controller that controls the coagulant dosing device.

In another aspect of the present disclosure, a method for coagulant dosing in a water treatment process comprises (a) collecting data relating to a combined content of insoluble and soluble ions in a source water stream at or upstream of a coagulant dosing device; (b) collecting data relating to a content of soluble ions in the source water stream downstream of the coagulant dosing device; (c) calculating, based on the data collected in steps (a) and (b), a soluble ion content-compensated turbidity of the source water stream at or upstream of the coagulant dosing device; and (d) commanding the coagulant dosing device to increase, decrease, or maintain a coagulant dosing rate to achieve a turbidity setpoint in the source water stream downstream of the coagulant dosing device.

In embodiments, step (a) may be carried out by a streaming current sensor.

In embodiments, step (a) may be carried out in a rapid mixing vessel of the water treatment system.

In embodiments, step (b) may be carried out by a streaming current sensor.

In embodiments, step (b) may be carried out by one or more sensors selected from the group consisting of an electrical conductivity analyzer, a total dissolved solids analyzer, an alkalinity analyzer, a UV254 analyzer, and combinations thereof.

In embodiments, step (b) may be carried out in a clear well of the water treatment system.

In embodiments, step (c) may comprise subtracting a second ion content value measured in step (b) from a first ion content value measured in step (a).

In embodiments, the data on which the calculation of step (c) is based may further comprise source water temperature data, source water pH data, sensor drift data, or a combination thereof.

In another aspect of the present disclosure, a streaming current monitoring system for a water treatment process comprises a computer; a first ion content sensor, positioned at or upstream of a coagulant dosing device of the water treatment process and configured to collect data relating to a combined content of insoluble and soluble ions in a source water stream; and a second ion content sensor, positioned downstream of the coagulant dosing device and configured to collect data relating to a content of soluble ions in the source water stream, wherein the computer comprises a processor and a non-transitory computer-readable medium storing instructions that, when executed by the processor, cause the processor to perform the steps of (a) calculating, based on the data collected by the first and second ion content sensors, a soluble ion content-compensated turbidity of the source water stream at or upstream of the coagulant dosing device; and (b) at least one of (i) displaying the soluble ion content-compensated turbidity of the source water stream in a graphical user interface of the computer; and (ii) communicating the soluble ion content-compensated turbidity of the source water stream to a controller that controls the coagulant dosing device.

In embodiments, the first ion content sensor may be a streaming current sensor.

In embodiments, the first ion content sensor may be positioned within a rapid mixing vessel of the water treatment process.

In embodiments, the second ion content sensor may be a streaming current sensor.

In embodiments, the second ion content sensor may be selected from the group consisting of an electrical conductivity analyzer, a total dissolved solids analyzer, an alkalinity analyzer, a UV254 analyzer, and combinations thereof.

In embodiments, the second ion content sensor may be positioned within a clear well of the water treatment process.

In embodiments, step (a) may comprise subtracting a second ion content value measured by the second ion content sensor from a first ion content value measured by the first ion content sensor.

In another aspect of the present disclosure, a method for calibration of a streaming current sensor comprises (a) dispensing a first streaming potential standard fluid, having a first known streaming current value, onto a surface of a streaming current sensor; (b) taking, by the streaming current sensor, a first streaming current reading corresponding to the first streaming potential standard fluid; (c) resetting a zero or offset value of the streaming current sensor based on a difference between the first known streaming current value and the first streaming current reading; (d) dispensing a second streaming potential standard fluid, having a second known streaming current value, onto the surface of the streaming current sensor; (e) taking, by the streaming current sensor, a second streaming current reading corresponding to the second streaming potential standard fluid; and (f) adjusting or recalibrating a sensitivity of the streaming current sensor based on a difference between the size of a range between the first and second known streaming potential values and the size of a range between the first and second streaming current readings.

In another aspect of the present disclosure, an automated coagulant dosing system for a water treatment process comprises a computer; a coagulant dosing device; a first ion content sensor, positioned at or upstream of the coagulant dosing device and configured to collect data relating to a combined content of insoluble and soluble ions in a source water stream; and a second ion content sensor, positioned downstream of the coagulant dosing device and configured to collect data relating to a content of soluble ions in the source water stream, wherein the computer comprises a processor and a non-transitory computer-readable medium storing instructions that, when executed by the processor, cause the processor to perform the steps of (a) calculating, based on the data collected by the first and second ion content sensors, a soluble ion content-compensated turbidity of the source water stream at or upstream of the coagulant dosing device; and (b) commanding the coagulant dosing device to increase, decrease, or maintain a coagulant dosing rate to achieve a turbidity setpoint in the source water stream downstream of the coagulant dosing device.

In embodiments, the first ion content sensor may be a streaming current sensor.

In embodiments, the first ion content sensor may be positioned within a rapid mixing vessel of the water treatment process.

In embodiments, the second ion content sensor may be a streaming current sensor.

In embodiments, the second ion content sensor may be selected from the group consisting of an electrical conductivity analyzer, a total dissolved solids analyzer, an alkalinity analyzer, a UV254 analyzer, and combinations thereof.

In embodiments, the second ion content sensor may be positioned within a clear well of the water treatment process.

In embodiments, step (a) may comprise subtracting a second ion content value measured by the second ion content sensor from a first ion content value measured by the first ion content sensor.

In embodiments, the automated chemical dosing system may further comprise at least one of a temperature sensor and a pH sensor.

In another aspect of the present disclosure, a streaming current sensor calibration system comprises a sensor chamber, surrounding and defining a sensor column and configured to receive and securely hold a streaming current sensor within the sensor column; a first container enclosing a first interior volume, wherein a first streaming potential standard fluid is contained within the first interior volume; and one or more calibration fluid dispensers, configured to dispense the first streaming potential standard fluid and a second streaming potential fluid onto a surface of the streaming current sensor to calibrate the streaming current sensor.

In embodiments, the streaming current sensor calibration system may further comprise a second container enclosing a second interior volume, wherein the second streaming potential standard fluid is contained within the second interior volume.

In embodiments, the streaming current sensor calibration system may further comprise a sample line in fluid communication with both a stream or vessel containing the second streaming potential standard fluid and the one or more calibration fluid dispensers and configured to convey the second streaming potential standard fluid from the stream or vessel containing the second streaming potential standard fluid to the one or more calibration fluid dispensers.

In embodiments, the one or more calibration fluid dispensers may consist of a single calibration fluid dispenser.

In embodiments, the one or more calibration fluid dispensers may comprise a first calibration fluid dispenser, configured to dispense the first streaming potential standard fluid onto the surface of the streaming current sensor, and a second calibration fluid dispenser, configured to dispense the second streaming potential standard fluid onto the surface of the streaming current sensor.

In embodiments, the first streaming potential standard fluid may comprise a suspension of latex beads in water.

In embodiments, the second streaming potential standard fluid may consist essentially of filtered or deionized water.

In another aspect of the present disclosure, a non-transitory computer-readable medium stores instructions that, when executed by a computer processor, cause the computer processor to perform a method comprising (a) collecting, from a first ion content sensor positioned at or upstream of a coagulant dosing device in a water treatment process, data relating to a combined content of insoluble and soluble ions in a source water stream; (b) collecting, from a second ion content sensor positioned downstream of the coagulant dosing device, data relating to a content of soluble ions in the source water stream; (c) calculating, based on the data collected in steps (a) and (b), a soluble ion content-compensated turbidity of the source water stream at or upstream of the coagulant dosing device; and (d) at least one of (i) displaying the soluble ion content-compensated turbidity of the source water stream in a graphical user interface and (ii) communicating the soluble ion content-compensated turbidity of the source water stream to a controller that controls the coagulant dosing device.

In another aspect of the present disclosure, a non-transitory computer-readable medium stores instructions that, when executed by a computer processor, cause the computer processor to perform a method comprising (a) collecting, from a first ion content sensor positioned at or upstream of a coagulant dosing device in a water treatment process, data relating to a combined content of insoluble and soluble ions in a source water stream; (b) collecting, from a second ion content sensor positioned downstream of the coagulant dosing device, data relating to a content of soluble ions in the source water stream; (c) calculating, based on the data collected in steps (a) and (b), a soluble ion content-compensated turbidity of the source water stream at or upstream of the coagulant dosing device; and (d) commanding the coagulant dosing device to increase, decrease, or maintain a coagulant dosing rate to achieve a turbidity setpoint in the source water stream downstream of the coagulant dosing device.

In another aspect of the present disclosure, a non-transitory computer-readable medium stores instructions that, when executed by a computer processor, cause the computer processor to perform a method comprising (a) commanding a fluid dispensing device to dispense a first streaming potential standard fluid, having a first known streaming current value, onto a surface of a streaming current sensor; (b) collecting, from the streaming current sensor, a first streaming current reading corresponding to the first streaming potential standard fluid; (c) resetting a zero or offset value of the streaming current sensor based on a difference between the first known streaming current value and the first streaming current reading; (d) commanding the same or a different fluid dispensing device to dispense a second streaming potential standard fluid, having a second known streaming current value, onto the surface of the streaming current sensor; (e) taking, by the streaming current sensor, a second streaming current reading corresponding to the second streaming potential standard fluid; and (f) adjusting or recalibrating a sensitivity of the streaming current sensor based on a difference between the size of a range between the first and second known streaming potential values and the size of a range between the first and second streaming current readings.

While specific embodiments and applications have been illustrated and described, the present disclosure is not limited to the precise configuration and components described herein. Various modifications, changes, and variations which will be apparent to those skilled in the art may be made in the arrangement, operation, and details of the methods and systems disclosed herein without departing from the spirit and scope of the overall disclosure.

As used herein, unless otherwise specified, the terms “about,” “approximately,” etc., when used in relation to numerical limitations or ranges, mean that the recited limitation or range may vary by up to 10%. By way of non-limiting example, “about 750” can mean as little as 675 or as much as 825, or any value therebetween. When used in relation to ratios or relationships between two or more numerical limitations or ranges, the terms “about,” “approximately,” etc. mean that each of the limitations or ranges may vary by up to 10%; by way of non-limiting example, a statement that two quantities are “approximately equal” can mean that a ratio between the two quantities is as little as 0.9:1.1 or as much as 1.1:0.9 (or any value therebetween), and a statement that a four-way ratio is “about 5:3:1:1” can mean that the first number in the ratio can be any value of at least 4.5 and no more than 5.5, the second number in the ratio can be any value of at least 2.7 and no more than 3.3, and so on.

The embodiments and configurations described herein are neither complete nor exhaustive. As will be appreciated, other embodiments are possible utilizing, alone or in combination, one or more of the features set forth above or described in detail below.

### BRIEF DESCRIPTION OF THE DRAWINGS

FIG. 1 is a schematic illustrating main components of a computerized system for calibration of streaming current measurement equipment, according to embodiments of the present disclosure.

FIG. 2 is a schematic illustrating a system for calibration and error compensation of streaming current measurement equipment, according to embodiments of the present disclosure.

FIG. 3 is a schematic illustrating main components of a system for calculating pH- and/or temperature-compensated streaming current values and communicating such information to an automated chemical dosing system in a water treatment process, according to embodiments of the present disclosure.

FIG. 4 is a schematic illustrating main components of a system for automated dosing of a coagulant in a water treatment process, according to embodiments of the present disclosure.

### DETAILED DESCRIPTION

Unless defined otherwise, all technical and scientific terms used herein have the same meaning as is commonly understood by one of ordinary skill in the art. All patents, applications, published applications, and other publications to which reference is made herein are incorporated by reference in their entirety. If there is a plurality of definitions for a term herein, the definition provided in the Summary prevails unless otherwise stated.

In the following description, for the purposes of explanation, numerous specific details are set forth in order to provide a thorough understanding of various embodiments disclosed herein. It will be apparent, however, to one skilled in the art that various embodiments of the present disclosure may be practiced without some of these specific details. The ensuing description provides exemplary embodiments only, and is not intended to limit the scope or applicability of the disclosure. Furthermore, to avoid unnecessarily obscuring the present disclosure, the preceding description omits a number of known structures and devices. This omission is not to be construed as a limitation of the scopes of the claims. Rather, the ensuing description of the exemplary embodiments will provide those skilled in the art with an enabling description for implementing an exemplary embodiment. It should however be appreciated that the present disclosure may be practiced in a variety of ways beyond the specific detail set forth herein.

For purposes of further disclosure and to comply with applicable written description and enablement requirements, the following references generally relate to systems and methods for water treatment and are hereby incorporated by reference in their entireties:

U.S. Patent Application Publication 2021/0010991, entitled “Sensor cleaning and calibration devices and systems,” published 14 Jan. 2021 to McLeod.

U.S. Patent Application Publication 2022/0298034, entitled “Automated methods and systems for optimized zeta potential chemical dosing in water treatment systems,” published 22 Sep. 2022 to McLeod.

In embodiments of the present disclosure, a streaming current monitoring system compensates “raw” measurements of the streaming current of a source water stream to account for a measured content of soluble ions in the source water stream. Particularly, the system includes both a streaming current sensor/analyzer with a small gain (i.e., high sensitivity), which obtains the “raw” streaming current measurements, and one or more other in-line sensors/analyzers adapted and configured to measure the soluble ion content of the source water stream; non-limiting examples of such sensors and analyzers include electrical conductivity analyzers, total dissolved solids analyzers, alkalinity analyzers, analyzers that measure the absorbance or transmittance of the water at an ultraviolet wavelength of 254 nanometers (UV254), and the like. Because the streaming current sensor/analyzer is set to a small-gain/high-sensitivity setting, it detects both the insoluble ion (i.e., turbidity-causing ion) content and the soluble ion content; systems of the present disclosure may therefore further include software that adjusts/compensates the readings from the streaming current sensor/analyzer based on the soluble ion readings from the other in-line sensors/analyzers to obtain a more accurate measurement of the “true” turbidity/insoluble ion content of the source water stream. The systems of the present disclosure can thus detect increases in turbidity that are coincident with decreases in soluble ion content (or vice versa), e.g., after rainstorms, snowmelt, changes in water source, etc.; such changes in the ion “mix” of the source water are invisible to presently available streaming current monitoring systems that rely on a low-gain/high-sensitivity streaming current sensor/analyzer alone. In some embodiments, the adjustment performed by the software may be as simple as subtracting the measured soluble ion content from the measured turbidity.

In embodiments of the present disclosure, a streaming current monitoring system compensates a first measurement of the streaming current of a source water stream to account for a second measurement of the streaming current of a source water stream, where the second measurement can reasonably be interpreted as measuring only the soluble ion content of the source water stream. By way of non-limiting example, many water treatment systems include a rapid mixing vessel, where coagulant is added to remove insoluble/turbidity-causing ions, and, downstream of the rapid mixing vessel, a “clear well,” i.e., a final storage basin where treated water is stored following filtration and disinfection to allow the disinfectant to inactivate any residual pathogens. Because the clear well is downstream of the rapid mixing vessel, i.e., downstream of the point where coagulant is added to remove insoluble ions and eliminate turbidity, the water in the clear well can be assumed to have no or negligible insoluble ions; as a result, a low-gain/high-sensitivity streaming current sensor/analyzer measuring the water in the clear well will detect only the soluble ion content of the stream. Systems of the present disclosure can therefore include software that uses a reading from a low-gain/high-sensitivity streaming current sensor/analyzer in the clear well to compensate the reading from a streaming current sensor/analyzer in (or upstream of) the rapid mixing vessel, which will detect both insoluble/turbidity-causing ions and soluble ions, to obtain a more accurate measurement of the “true” turbidity/insoluble ion content of the incoming source water stream. In some embodiments, the adjustment performed by the software may be as simple as subtracting the measured streaming current in the clear well from the measured streaming current in (or upstream of) the rapid mixing vessel. In some embodiments, the streaming current monitoring system may further include pH and/or temperature sensors in (or upstream of) the rapid mixing vessel, in the clear well, or both; readings from the pH and/or temperature measurements may be used by the software to recalibrate the streaming current sensors/analyzers (e.g., by compensating for variations in pH/temperature of the source water from the pH/temperature of “standard” fluids on which the streaming current sensors/analyzers are initially calibrated, which may otherwise introduce errors into the measured streaming current values) and/or confirm the accuracy of the readings therefrom. In some embodiments, the streaming current sensor/analyzer in or upstream of the rapid mixing vessel may have a higher gain/lower sensitivity than the streaming current sensor/analyzer in the clear well.

In embodiments of the present disclosure, an automated coagulant dosing system optimizes metal salt coagulant dosing for the mitigation or elimination of source water turbidity. In these embodiments, one or more proportional-integral-derivative (PID) loop(s) maintain(s) a turbidity setpoint (which in many embodiments may be zero, or close to zero, e.g., 0.5 nephelometric turbidity units (NTU)) by controlled dosing of coagulant into the source water, and can make dynamic, real-time adjustments to the coagulant dose based on fluctuations in a soluble ion-compensated measurement of the turbidity of the incoming source water. As described above in relation to the streaming current monitoring systems of this disclosure, the one or more PID loop(s) take(s) into account readings from at least two sensors/analyzers, which may in embodiments comprise a streaming current sensor/analyzer and a sensor/analyzer that measures soluble ion content directly (e.g., an electrical conductivity analyzer, a total dissolved solids analyzer, an alkalinity analyzer, a UV254 analyzer, etc.) or a first streaming current sensor/analyzer positioned at or upstream of the point where coagulant is dosed (e.g., in a rapid mixing vessel) and a second streaming current sensor/analyzer positioned downstream of the point where coagulant is dosed (e.g., in a clear well), to determine the soluble ion-compensated measurement of the turbidity of the incoming source water. Upon detecting a change in this compensated turbidity measurement, the one or more PID loop(s) command(s) a coagulant dosing device (e.g., a pump) to increase or decrease the rate at which coagulant is added to the source water to maintain the turbidity setpoint. In this way, the coagulant dosing systems of the disclosure may offset changes in the buffering capacity of the water, in much the same way that systems and devices for controlling the pH of a source water stream can achieve a desired pH value by varying the rate of acid or base dosing when the buffering capacity of the water changes. In some embodiments, the coagulant dosing systems of the disclosure may allow an operator or technician to adjust the turbidity setpoint and/or the underlying control logic, based on source water quality and other factors.

In embodiments of the present disclosure, a dual-chemical reagent sensor calibration system allows a streaming current sensor/analyzer to be quickly (1) withdrawn from service measuring the streaming potential of a source water stream, (2) recalibrated against standard fluids having a known zeta potential/streaming current (e.g., to eliminate or correct measurement “drift” of the sensor/analyzer), and (3) returned to service measuring the streaming potential of the source water stream. In general, such systems may be effective to (re)calibrate a streaming current sensor/analyzer by methods similar to those used to (re)calibrate pH sensors. Particularly, systems according to such embodiments include a sealed container that holds a standard fluid (e.g., deionized water with latex beads suspended therein) having a known and stable zeta potential (e.g., −40 mV) and/or streaming current; such standard fluids are known in the art and readily commercially available. In these embodiments, the streaming current sensor/analyzer may be isolated from the source water stream by any suitable means, drained and/or rinsed, and exposed to a sample of the standard fluid (e.g., a sample of the standard fluid can be directed from the sealed container into a column or housing within which the streaming current sensor/analyzer is disposed. Software of the dual-chemical reagent sensor calibration system (or another system associated therewith, e.g., a streaming current monitoring and/or coagulant dosing system as described elsewhere throughout this disclosure) can then compare the streaming current reading taken by the sensor/analyzer against the known streaming current of the standard fluid and adjust/recalibrate the zero value of the sensor/analyzer such that the two readings match, thereby mitigating or eliminating the “zero drift” or “offset drift” (i.e., consistent shift across all measured values) of the sensor/analyzer. The dual-chemical reagent sensor calibration system further comprises a bath or stream of another calibration standard fluid having a different known zeta potential/streaming current (e.g., a filtered/deionized water having a zeta potential of zero or immeasurably different from zero), which may in embodiments include a volume of the fluid inside a second, separate sealed container and/or a sample line from a stream of filtered/deionized water (e.g., an outlet stream of the water treatment process). After elimination of zero/offset drift as described above, the sensor/analyzer is transferred (optionally after a second rinsing/draining to remove the residual first calibration fluid) to the bath or stream of the second calibration fluid; the same software as above can then compare the streaming current reading taken by the sensor/analyzer against the known streaming current of the second standard fluid and adjust/recalibrate the sensitivity of the sensor/analyzer such that the two readings match, thereby mitigating or eliminating the “span drift” (i.e., proportional increasing or decreasing shift of the measured value away from the calibrated value as the measured value increases or decreases) of the sensor/analyzer. The recalibrated streaming current sensor/analyzer can then be returned to service measuring a streaming current in the source water stream (optionally after a second or third rinsing/draining to remove the residual second calibration fluid). In some embodiments, the volume(s) of the first (and, as the case may be, second) calibration fluid(s) may be housed in one or more “bag-in-box” (BiB) container(s), e.g., Cubitainer(s), of any suitable type as may be known in the art, which may in turn be part of, by way of non-limiting example, a shelf assembly. In some embodiments, the dual-chemical reagent sensor calibration system may further comprise temperature sensors to compensate for temperature differences.

In embodiments of the present disclosure, a water treatment process or system includes any one, any two, or all three of a streaming current monitoring system as disclosed herein, an automated coagulant dosing system as disclosed herein, and a dual-chemical reagent sensor calibration system as disclosed herein. Particularly, a water treatment process may include at least two sensors, which may in embodiments comprise a streaming current sensor/analyzer and a sensor/analyzer that measures soluble ion content directly (e.g., an electrical conductivity analyzer, a total dissolved solids analyzer, an alkalinity analyzer, a UV254 analyzer, etc.) or a first streaming current sensor/analyzer positioned at or upstream of the point where coagulant is dosed (e.g., in a rapid mixing vessel) and a second streaming current sensor/analyzer positioned downstream of the point where coagulant is dosed (e.g., in a clear well), to determine the soluble ion-compensated measurement of the turbidity of the incoming source water. Either or both of these two sensors may be provided in association with a dual-chemical reagent sensor calibration system as disclosed herein (if both sensors are provided in association with such a system, they may be provided in association with the same such system or two separate such systems), such that either or both of these sensors may be recalibrated from time to time and/or such that the water treatment process or system (or software/control logics associated implemented thereby) can compensate for measurement error/drift from either or both of the sensors.

In systems according to the present disclosure, either or both of the two sensors described above may be calibrated and/or compensated by methods/systems other than the dual-chemical reagent methods and systems described above, and/or other sensors or data may be used to place limits on the amount of coagulant dosing if the streaming current readings obtained by the sensors are “out of range.” By way of first non-limiting example, one embodiment of a streaming current monitoring system according to the present disclosure includes automatic titration equipment that obtains a highly accurate measurement of the alkalinity of the source water stream at one or more specified time points and places upper and lower bounds on the compensated streaming current value of the source water based on this measured alkalinity (i.e., correcting any “raw” streaming current reading, by one or more streaming current sensor(s)/analyzer(s), that falls below the lower bound to a compensated value that is equal to the lower bound, or a “raw” streaming current reading that exceeds the upper bound to a compensated value that is equal to the upper bound), as streaming current/zeta potential readings may be exaggerated in highly alkaline waters. By way of second non-limiting example, an embodiment of a streaming current monitoring and/or automated coagulant dosing system according to the present disclosure includes water hardness testing equipment (whether automatic, manual, or both) that obtains a highly accurate measurement of the total dissolved minerals or salts in the source water stream at one or more specified time points, such that software of the system (or a control logic implemented thereby) places upper and lower bounds on the compensated streaming current value of the source water (as described above) and/or the allowable coagulant dosing rate based on this measured water hardness.

In some embodiments of the present disclosure, any one or more PID loops of a streaming current monitoring system or a control logic thereof may determine the relationship between the “raw” measured turbidity value(s) and the soluble ion-compensated turbidity value(s) according to a deterministic equation in which the various input variables are used as weights, coefficients, or the like. Additionally or alternatively, any one or more PID loops of the streaming current monitoring system or a control logic thereof may determine the relationship between the “raw” measured turbidity value(s) and the soluble ion-compensated turbidity value(s) by use of a lookup table stored in a computer memory; by way of non-limiting example, a computer memory may contain a lookup table that stores a set of equations or other mathematical relationships that define the relationship between “raw” and soluble-ion adjusted turbidity values at each of several different water temperatures, and the PID loop or control logic thereof may look up the equations or other mathematical relationships based on a water temperature measurement received from a water temperature sensor. Additionally or alternatively, any one or more PID loops of the streaming current monitoring system or a control logic thereof may determine the relationship between the “raw” measured turbidity value(s) and the soluble ion-compensated turbidity value(s) by employing any one or more machine learning algorithms, using historical water treatment input variables and treatment outcomes as training data to construct equations and/or weight input variables in an iterative manner, which may enable more complex, complete, and/or nuanced relationships between input variables and water treatment outcomes. Machine learning algorithms employed by the PID loop or control logic thereof may be either supervised or unsupervised and may include, by way of non-limiting example, TensorFlow, NaiveBayes, Logistic Regression, and Random Forest. Machine learning algorithms may allow for soluble ion compensation of measured raw turbidity values to be more readily adjusted dynamically and in real time in response to source water variability (e.g., spikes in turbidity and concurrent decreases in soluble ion content after storm events). The machine learning algorithm may impose upper and/or lower limits on the soluble ion-compensated turbidity value(s) to prevent overdosing or underdosing of coagulant based on historical data (e.g., pH, temperature, identity of coagulant, etc.).

While the exemplary aspects, embodiments, and/or configurations illustrated herein show the various components of the system collocated, certain components of the system can be located remotely, at distant portions of a distributed network, such as a LAN and/or the Internet, or within a dedicated system. Thus, it should be appreciated, that the components of the system can be combined in to one or more devices or collocated on a particular node of a distributed network, such as an analog and/or digital telecommunications network, a packet-switch network, or a circuit-switched network. It will be appreciated from the following description, and for reasons of computational efficiency, that the components of the system can be arranged at any location within a distributed network of components without affecting the operation of the system.

Furthermore, it should be appreciated that the various links connecting the elements can be wired or wireless links, or any combination thereof, or any other known or later developed element(s) that is capable of supplying and/or communicating data to and from the connected elements. These wired or wireless links can also be secure links and may be capable of communicating encrypted information. Transmission media used as links, for example, can be any suitable carrier for electrical signals, including coaxial cables, copper wire and fiber optics, and may take the form of acoustic or light waves, such as those generated during radio-wave and infra-red data communications.

As used herein, the phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B and C together.

The term “a” or “an” entity refers to one or more of that entity. As such, the terms “a” (or “an”), “one or more” and “at least one” can be used interchangeably herein. It is also to be noted that the terms “comprising,” “including,” and “having” can be used interchangeably.

The term “automatic” and variations thereof, as used herein, refers to any process or operation done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

The term “computer-readable medium” as used herein refers to any tangible storage and/or transmission medium that participate in providing instructions to a processor for execution. Such a medium may take many forms, including but not limited to, non-volatile media, volatile media, and transmission media. Non-volatile media includes, for example, NVRAM, or magnetic or optical disks. Volatile media includes dynamic memory, such as main memory. Common forms of computer-readable media include, for example, a floppy disk, a flexible disk, hard disk, magnetic tape, or any other magnetic medium, magneto-optical medium, a CD-ROM, any other optical medium, punch cards, paper tape, any other physical medium with patterns of holes, a RAM, a PROM, and EPROM, a FLASH-EPROM, a solid state medium like a memory card, any other memory chip or cartridge, a carrier wave as described hereinafter, or any other medium from which a computer can read. A digital file attachment to e-mail or other self-contained information archive or set of archives is considered a distribution medium equivalent to a tangible storage medium. When the computer-readable media is configured as a database, it is to be understood that the database may be any type of database, such as relational, hierarchical, object-oriented, and/or the like. Accordingly, the disclosure is considered to include a tangible storage medium or distribution medium and prior art-recognized equivalents and successor media, in which the software implementations of the present disclosure are stored.

A “computer readable signal” medium may include a propagated data signal with computer readable program code embodied therein, for example, in baseband or as part of a carrier wave. Such a propagated signal may take any of a variety of forms, including, but not limited to, electromagnetic, optical, or any suitable combination thereof. A computer readable signal medium may be any computer readable medium that is not a computer readable storage medium and that can communicate, propagate, or transport a program for use by or in connection with an instruction execution system, apparatus, or device. Program code embodied on a computer readable medium may be transmitted using any appropriate medium, including but not limited to wireless, wireline, optical fiber cable, RF, etc., or any suitable combination of the foregoing.

The terms “determine,” “calculate,” and “compute,” and variations thereof, as used herein, are used interchangeably and include any type of methodology, process, mathematical operation or technique.

It shall be understood that the term “means” as used herein shall be given its broadest possible interpretation in accordance with 35 U.S.C. § 112(f). Accordingly, a claim incorporating the term “means” shall cover all structures, materials, or acts set forth herein, and all of the equivalents thereof. Further, the structures, materials or acts and the equivalents thereof shall include all those described in the summary of the disclosure, brief description of the drawings, detailed description, abstract, and claims themselves.

Aspects of the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Any combination of one or more computer readable medium(s) may be utilized. The computer readable medium may be a computer readable signal medium or a computer readable storage medium.

In yet another embodiment, the systems and methods of this disclosure can be implemented in conjunction with a special purpose computer, a programmed microprocessor or microcontroller and peripheral integrated circuit element(s), an ASIC or other integrated circuit, a digital signal processor, a hard-wired electronic or logic circuit such as discrete element circuit, a programmable logic device or gate array such as PLD, PLA, FPGA, PAL, special purpose computer, any comparable means, or the like. In general, any device(s) or means capable of implementing the methodology illustrated herein can be used to implement the various aspects of this disclosure. Exemplary hardware that can be used for the disclosed embodiments, configurations, and aspects includes computers, handheld devices, telephones (e.g., cellular, Internet enabled, digital, analog, hybrids, and others), and other hardware known in the art. Some of these devices include processors (e.g., a single or multiple microprocessors), memory, nonvolatile storage, input devices, and output devices. Furthermore, alternative software implementations including, but not limited to, distributed processing or component/object distributed processing, parallel processing, or virtual machine processing can also be constructed to implement the methods described herein.

Examples of the processors as described herein may include, but are not limited to, at least one of Qualcomm® Snapdragon® 800 and 801, Qualcomm® Snapdragon® 610 and 615 with 4G LTE Integration and 64-bit computing, Apple® A7 processor with 64-bit architecture, Apple® M7 motion coprocessors, Samsung® Exynos® series, the Intel® Core™ family of processors, the Intel® Xeon® family of processors, the Intel® Atom™ family of processors, the Intel Itanium® family of processors, Intel® Core® i5-4670K and i7-4770K 22 nm Haswell, Intel® Core® i5-3570K 22 nm Ivy Bridge, the AMD® FX™ family of processors, AMD® FX-4300, FX-6300, and FX-8350 32 nm Vishera, AMD® Kaveri processors, Texas Instruments® Jacinto C6000™ automotive infotainment processors, Texas Instruments® OMAP™ automotive-grade mobile processors, ARM® Cortex™-M processors, ARM® Cortex-A and ARM926EJ-S™ processors, other industry-equivalent processors, and may perform computational functions using any known or future-developed standard, instruction set, libraries, and/or architecture.

In yet another embodiment, the disclosed methods may be readily implemented in conjunction with software using object or object-oriented software development environments that provide portable source code that can be used on a variety of computer or workstation platforms. In additional embodiments, the disclosed methods may be implemented in conjunction with functional programming. Alternatively, the disclosed system may be implemented partially or fully in hardware using standard logic circuits or VLSI design. Whether software or hardware is used to implement the systems in accordance with this disclosure is dependent on the speed and/or efficiency requirements of the system, the particular function, and the particular software or hardware systems or microprocessor or microcomputer systems being utilized.

In yet another embodiment, the disclosed methods may be partially implemented in software that can be stored on a storage medium, executed on programmed general-purpose computer with the cooperation of a controller and memory, a special purpose computer, a microprocessor, or the like. In these instances, the systems and methods of this disclosure can be implemented as program embedded on personal computer such as an applet, JAVA® or CGI script, as a resource residing on a server or computer workstation, as a routine embedded in a dedicated measurement system, system component, or the like. The system can also be implemented by physically incorporating the system and/or method into a software and/or hardware system.

Although the present disclosure describes components and functions implemented in the aspects, embodiments, and/or configurations with reference to particular standards and protocols, the aspects, embodiments, and/or configurations are not limited to such standards and protocols. Other similar standards and protocols not mentioned herein are in existence and are considered to be included in the present disclosure. Moreover, the standards and protocols mentioned herein, and other similar standards and protocols not mentioned herein are periodically superseded by faster or more effective equivalents having essentially the same functions. Such replacement standards and protocols having the same functions are considered equivalents included in the present disclosure.

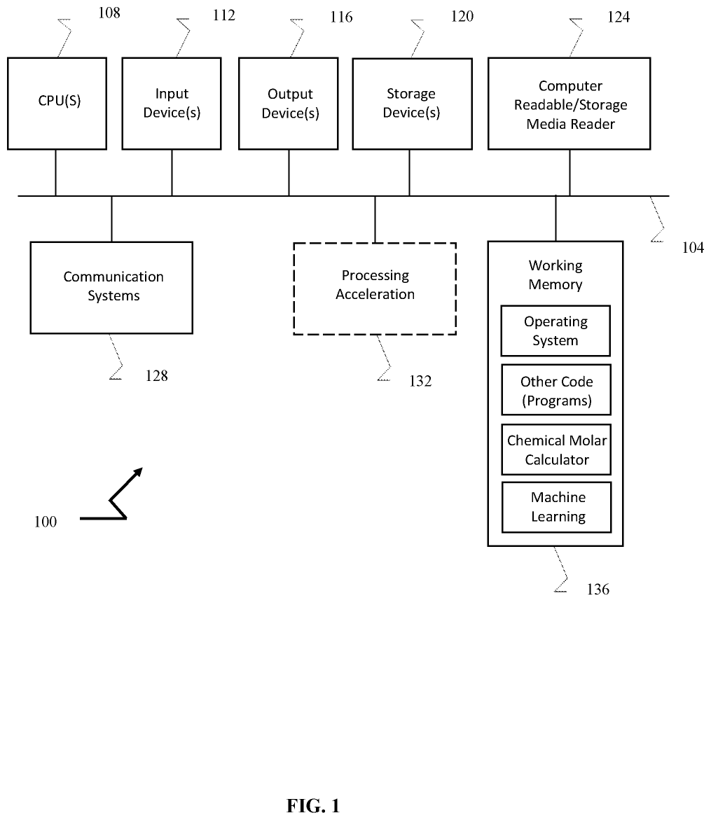

FIG. 1 is a block diagram illustrating elements of an exemplary computing device in which embodiments of the present disclosure may be implemented. More specifically, this example illustrates one embodiment of a computer system 100 upon which the servers, user computers, computing devices, or other systems or components described above may be deployed or executed. The computer system 100 is shown comprising hardware elements that may be electrically coupled via a bus 104. The hardware elements may include one or more Central Processing Units (CPUs) 108, which may in embodiments include any one or more programmable logic controllers (PLCs), microprocessors, or the like; one or more input devices 112, which may in embodiments include one or more user input devices (e.g., a mouse, a keyboard, etc.) and/or one or more sensors or analyzers (e.g. a flow rate sensor, a turbidity sensor, a pH sensor, a temperature sensor, an organics sensor, etc.); and one or more output devices 116, which may in embodiments include one or more devices for outputting information in a manner intelligible by a human technician (e.g., a display device, a printer, etc.) and/or one or more output devices or signals that may command and/or control components of a water treatment process (e.g., chemical dosing pumps, delay features, speed controls, proportional-integral-derivative (PID) controllers, and the like). The computer system 100 may also include one or more storage devices 120. By way of example, storage device(s) 120 may be disk drives, optical storage devices, solid-state storage devices such as a Random-Access Memory (RAM) and/or a Read-Only Memory (ROM), which can be programmable, flash-updateable and/or the like.

The computer system 100 may additionally include a computer-readable storage media reader 124; a communications system 128 (e.g., a modem, a network card (wireless or wired), an infra-red communication device, etc.); and working memory 136, which may include RAM and ROM devices as described above. The computer system 100 may also include a processing acceleration unit 132, which can include a Digital Signal Processor (DSP), a special-purpose processor, and/or the like.

The computer-readable storage media reader 124 can further be connected to a computer-readable storage medium, together (and, optionally, in combination with storage device(s) 120) comprehensively representing remote, local, fixed, and/or removable storage devices plus storage media for temporarily and/or more permanently containing computer-readable information. The communications system 128 may permit data to be exchanged with a network and/or any other computer described above with respect to the computer environments described herein. Moreover, as disclosed herein, the term “storage medium” may represent one or more devices for storing data, including ROM, RAM, magnetic RAM, core memory, magnetic disk storage mediums, optical storage mediums, flash memory devices and/or other machine-readable mediums for storing information.

The computer system 100 may also comprise software elements, shown as being currently located within a working memory 136, including an operating system, a chemical molar calculator, a machine learning algorithm, and/or other code or programs. It should be appreciated that alternate embodiments of a computer system 100 may have numerous variations from that described above. For example, customized hardware might also be used and/or particular elements might be implemented in hardware, software (including portable software, such as applets), or both. Further, connection to other computing devices such as network input/output devices may be employed.

Examples of the processors 108 as described herein may include, but are not limited to, at least one of Qualcomm® Snapdragon® 800 and 801, Qualcomm® Snapdragon® 620 and 615 with 4G LTE Integration and 64-bit computing, Apple® A7 processor with 64-bit architecture, Apple® M7 motion coprocessors, Samsung® Exynos® series, the Intel® Core™ family of processors, the Intel® Xeon® family of processors, the Intel® Atom™ family of processors, the Intel Itanium® family of processors, Intel® Core® i5-4670K and i7-4770K 22 nm Haswell, Intel® Core® i5-3570K 22 nm Ivy Bridge, the AMD® FX™ family of processors, AMD® FX-4300, FX-6300, and FX-8350 32 nm Vishera, AMD® Kaveri processors, Texas Instruments® Jacinto C6000™ automotive infotainment processors, Texas Instruments® OMAP™ automotive-grade mobile processors, ARM® Cortex™-M processors, ARM® Cortex-A and ARM926EJ-S™ processors, other industry-equivalent processors, and may perform computational functions using any known or future-developed standard, instruction set, libraries, and/or architecture.

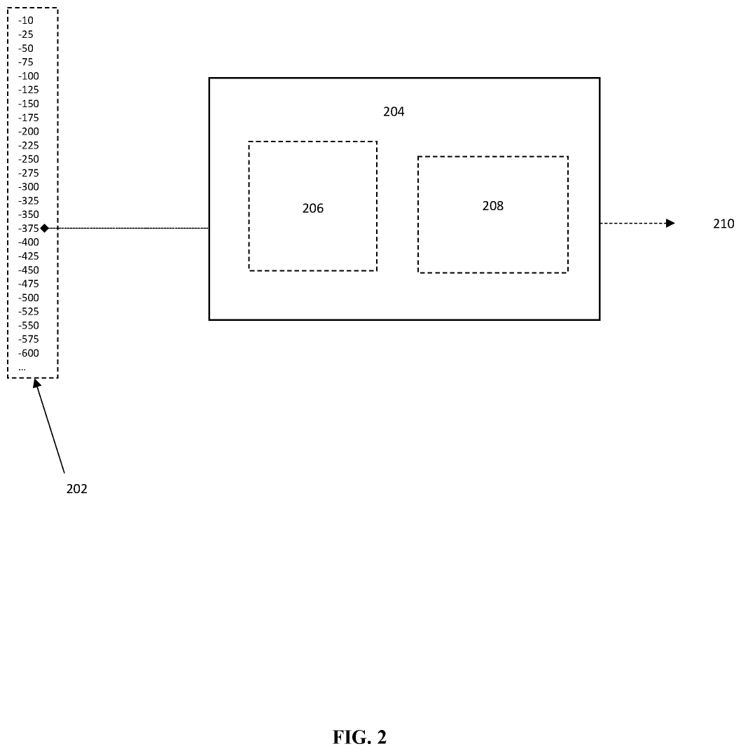

Referring now to FIG. 2, an embodiment of a streaming current monitoring system and method 200 in a water treatment process is illustrated. In the control scheme illustrated in FIG. 2, a programmable logic controller (PLC) 204 continually receives raw streaming current data 202, e.g., raw readings from a low-gain/high-sensitivity streaming current sensor/analyzer (not shown). The PLC embodies software that applies either or both of two compensation algorithms, a dissolved ion content compensation algorithm 206 and a temperature and/or calibration compensation algorithm 208.

Upon receiving the raw streaming current data 202, the PLC 204 applies the dissolved ion content compensation algorithm 206 to adjust the raw streaming current data 202 for soluble ions that may have been detected by the streaming current analyzer/sensor in addition to insoluble/turbidity-causing ions; as described elsewhere throughout this disclosure, this adjustment is based on data relating to the soluble ion content of the source water stream (e.g., any one or more of total dissolved solids data, electrical conductivity data, alkalinity data, water hardness data, UV254 data, and the like). It is to be expressly understood that the data relating to the soluble ion content upon which the adjustment applied by the dissolved ion content compensation algorithm 206 is based may in some embodiments be continual and/or real time data (e.g., data continually or frequently communicated to the PLC 204 by a total dissolved solids analyzer/sensor, an electrical conductivity sensor/analyzer, an alkalinity sensor/analyzer, a water hardness sensor/analyzer, a UV254 sensor/analyzer, etc.), whereas in other embodiments (e.g., where continual measurement/monitoring of soluble ions in the source water stream is impractical or impossible) these data may simply be references values or assumed values stored in a computer memory of the PLC 204. Regardless of the source and type of the data relating to the soluble ion content of the source water, the PLC 204, in applying the dissolved ion content compensation algorithm 206, may, in embodiments, utilize a lookup table, equation, and/or machine learning algorithm to determine how to compensate the raw streaming current data 202 for the soluble ion content of the source water stream.

The PLC 204 also applies the temperature and/or calibration compensation algorithm 208 to adjust the raw streaming current data 202 for temperature and/or calibration effects; it is to be expressly understood that the PLC 204 may apply the dissolved ion content compensation algorithm 206 and the temperature and/or calibration compensation algorithm 208 simultaneously or sequentially in any order. The temperature and/or calibration compensation algorithm 208 may in some embodiments adjust for variations in source water temperature not accounted for by the dissolved ion content compensation algorithm 206. By way of non-limiting example, the adjustment applied by the dissolved ion content compensation algorithm 206 may be based upon a relationship between raw measured turbidity values and soluble ion content-adjusted turbidity values that is empirically known but valid only at a certain temperature (or within a certain temperature range); the temperature and/or calibration compensation algorithm 208 may modify this relationship where the source water temperature differs from the temperature(s) at which the relationship embodied in the dissolved ion content compensation algorithm 206 is known to be valid. Additionally or alternatively, the temperature and/or calibration compensation algorithm 208 may in some embodiments adjust for a known error or “drift” of a streaming current sensor/analyzer that collects the raw streaming current data 202; by way of non-limiting example, the temperature and/or calibration compensation algorithm 208 may compensate for offset drift and/or span drift assessed by a sensor calibration system, such as a dual-chemical reagent sensor calibration system as described elsewhere throughout this disclosure. It is to be expressly understood that data relating to the source water temperature and/or sensor calibration upon which the adjustment applied by the temperature and/or calibration compensation algorithm 208 is based may in some embodiments be continual and/or real time data (e.g., data continually or frequently communicated to the PLC 204 by a temperature sensor, measurements of sensor error/drift that are updated by a sensor calibration system on a continual or recurring basis, etc.), whereas in other embodiments (e.g., where continual or repeated measurement/monitoring of source water temperature and/or sensor error/drift is impractical or impossible) these data may simply be references values or assumed values stored in a computer memory of the PLC 204. Regardless of the source and type of the data relating to the source water temperature and/or sensor calibration, the PLC 204, in applying the temperature and/or calibration compensation algorithm 208, may, in embodiments, utilize a lookup table, equation, and/or machine learning algorithm to determine how to compensate the raw streaming current data 202 for the temperature of the source water stream and/or the error/drift of the streaming current sensor/analyzer(s) that collect(s) the raw streaming current data 202.

Application of the dissolved ion content compensation algorithm 206 and the temperature and/or calibration compensation algorithm 208 by the PLC 204 results in the computation of a compensated streaming current value 210, which the PLC 204 may then communicate to other components of the water treatment process in which the streaming current monitoring system and method 200 is embodied (e.g., to adjust a coagulant dosing rate).

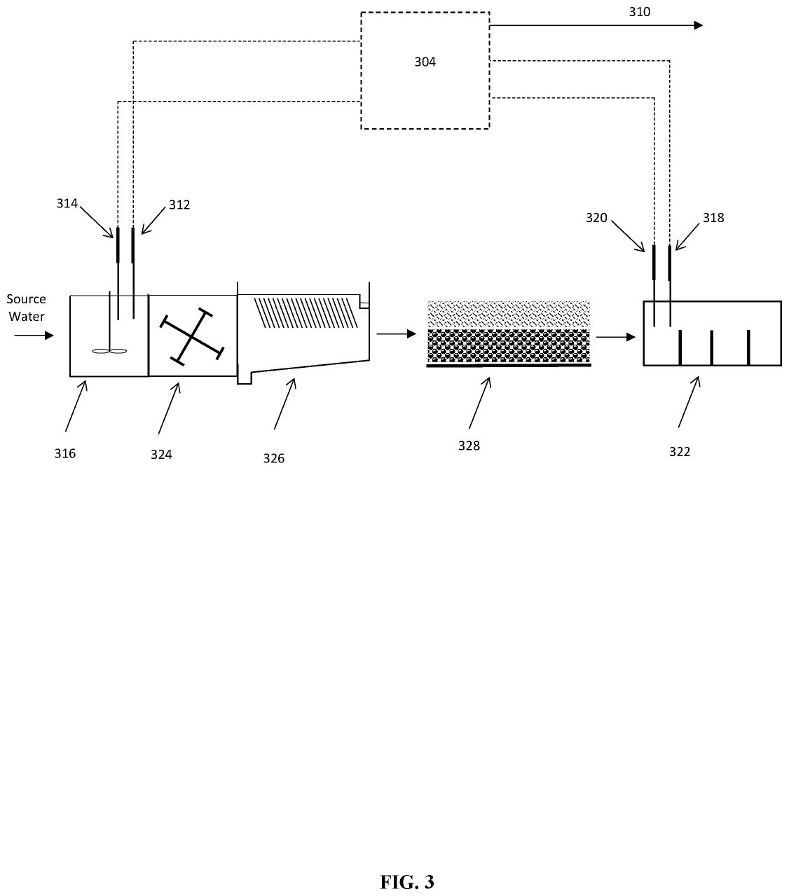

Referring now to FIG. 3, a streaming current monitoring system 300 according to the present disclosure is illustrated. As shown in FIG. 3, the streaming current monitoring system 300 comprises a computer 304 that receives data from a first streaming current sensor/analyzer 312 and (optionally) first pH and/or temperature sensor(s) 314 disposed within a rapid mixing vessel 316, and further receives data from a second streaming current sensor/analyzer 318 and (optionally) second pH and/or temperature sensor(s) 320 downstream of the rapid mixing vessel 316. While FIG. 3 illustrates the second streaming current sensor/analyzer 318 and optional second pH and/or temperature sensor(s) 320 as located in a clear well 322, it is to be expressly understood that these sensors may suitably be located at any point downstream of the point where coagulant has been added to the source water and the coagulated insoluble ions have been allowed to precipitate therefrom. At least the second streaming current sensor/analyzer 318, and in many embodiments both streaming current sensors/analyzers 312,318, operate on a low-gain/high-sensitivity setting such that they detect both suspended/insoluble (i.e., turbidity-causing) ions and soluble ions. In general, coagulant is added to the source water in the rapid mixing vessel 316 or a maturation mixing vessel 324 immediately downstream thereof; the source water then flows into a clarifier 326, where the coagulated turbidity-causing particles present in the source water may floc and precipitate out of the source water. The source water subsequently flows through a multimedia filter 328 to filter remaining particulates and finally flows into the clear well 322 as a final storage step (typically to allow chemical disinfectants to eliminate any biological pathogens in the source water). Thus, as described elsewhere throughout this disclosure, the second streaming current sensor/analyzer 318, which as noted above is positioned in the clear well 322 or at any other point downstream of the point where insoluble ions floc and precipitate out of the source water, takes streaming current readings of the source water after the desired turbidity setpoint has been achieved and therefore detects only soluble ions (plus whatever concentration of insoluble ions is considered acceptable based on the selected turbidity setpoint).

Based on the data received from the first and second streaming current sensors/analyzers 312,318, and optionally the first and second pH and/or temperature sensor(s) 314,320, the computer 304 determines a soluble ion-compensated streaming current value that corresponds to a “true,” or at the very least much more accurate, turbidity measurement of the source water prior to addition of the metal salt coagulant. In some embodiments, the adjustment performed by the computer 304 may be as simple as subtracting the reading obtained by the second streaming current sensor/analyzer 318 from the reading obtained by the first streaming current sensor/analyzer 312. As further described, for example, with reference to FIG. 2, the computer 304 may, in some embodiments, also correct readings obtained from the first streaming current sensor/analyzer 312 for source water pH, source water temperature, known sensor/calibration errors, etc. The computer 304 then communicates a compensated streaming current value 310 to other components of the water treatment process in which the streaming current monitoring system 300 is embodied (e.g., to adjust a coagulant dosing rate). The streaming current monitoring system 300 may include other features that are not illustrated in FIG. 3, such as, by way of non-limiting example, one or more user interface devices (which may in embodiments communicate information relating to the raw and/or compensated streaming values, by auditory and/or visual means, to a technician or operator) and/or one or more additional sensors for measuring additional parameters of the source water.

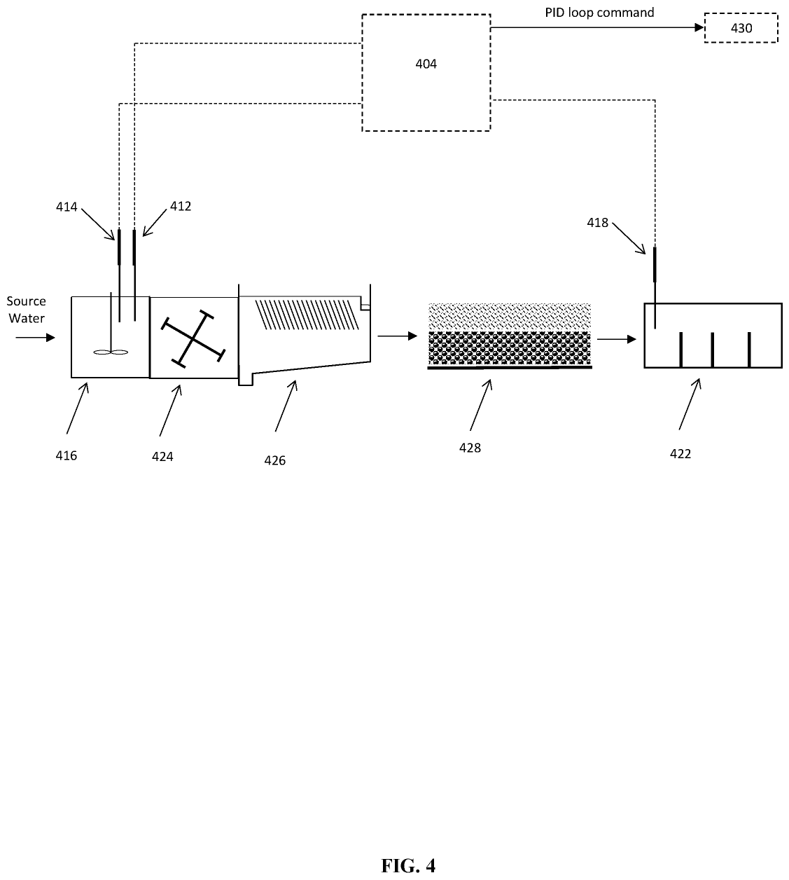

Referring now to FIG. 4, an automated coagulant dosing system 400 according to the present disclosure is illustrated. As shown in FIG. 4, the automated coagulant dosing system 400 comprises a computer 404 that receives data from a first streaming current sensor/analyzer 412 and (optionally) first pH and/or temperature sensor(s) 414 disposed within a rapid mixing vessel 416, and further receives data from a second streaming current sensor/analyzer 418 and (optionally) second pH and/or temperature sensor(s) (not shown) downstream of the rapid mixing vessel 416. While FIG. 4 illustrates the second streaming current sensor/analyzer 418 as located in a clear well 422, it is to be expressly understood that these sensors may suitably be located at any point downstream of the point where coagulant has been added to the source water and the coagulated insoluble ions have been allowed to precipitate therefrom. At least the second streaming current sensor/analyzer 418, and in many embodiments both streaming current sensors/analyzers 412,418, operate on a low-gain/high-sensitivity setting such that they detect both suspended/insoluble (i.e., turbidity-causing) ions and soluble ions. In general, coagulant is added to the source water in the rapid mixing vessel 416 or a maturation mixing vessel 424 immediately downstream thereof; the source water then flows into a clarifier 426, where the coagulated turbidity-causing particles present in the source water may floc and precipitate out of the source water. The source water subsequently flows through a multimedia filter 428 to filter remaining particulates and finally flows into the clear well 422 as a final storage step (typically to allow chemical disinfectants to eliminate any biological pathogens in the source water). Thus, as described elsewhere throughout this disclosure, the second streaming current sensor/analyzer 418, which as noted above is positioned in the clear well 422 or at any other point downstream of the point where insoluble ions floc and precipitate out of the source water, takes streaming current readings of the source water after the desired turbidity setpoint has been achieved and therefore detects only soluble ions (plus whatever concentration of insoluble ions is considered acceptable based on the selected turbidity setpoint).

Based on the data received from the first and second streaming current sensors/analyzers 412,418, and optionally the first pH and/or temperature sensor(s) 414 and second pH and/or temperature sensor(s), the computer 404 determines a soluble ion-compensated streaming current value that corresponds to a “true,” or at the very least much more accurate, turbidity measurement of the source water prior to addition of the metal salt coagulant. In some embodiments, the adjustment performed by the computer 404 may be as simple as subtracting the reading obtained by the second streaming current sensor/analyzer 418 from the reading obtained by the first streaming current sensor/analyzer 412. As further described, for example, with reference to FIG. 2, the computer 404 may, in some embodiments, also correct readings obtained from the first streaming current sensor/analyzer 412 for source water pH, source water temperature, known sensor/calibration errors, etc. The automated coagulant dosing system 400 may include other features that are not illustrated in FIG. 4, such as, by way of non-limiting example, one or more user interface devices (which may in embodiments communicate information relating to the raw and/or compensated streaming values, by auditory and/or visual means, to a technician or operator) and/or one or more additional sensors for measuring additional parameters of the source water.

The difference between the streaming current monitoring system 300 illustrated in FIG. 3 and the automated coagulant dosing system 400 illustrated in FIG. 4 is that, rather than simply calculating compensated insoluble ion content values and communicating these values to separate components that control water treatment process operations, as the computer 300 of the streaming current monitoring system 300 does, the computer 404 of the automated coagulant dosing system 400 controls certain of these operations, particularly coagulant dosing, directly. Specifically, having calculated a compensated streaming current value, the computer 404 then acts as a controller of a proportional-integral-derivative (PID) control loop that regulates a coagulant dosing device 430 to achieve and maintain a turbidity setpoint (which in many embodiments may be zero or close to zero, e.g., about 0.5 NTU) in the clear well 422 or otherwise downstream of the rapid mixing vessel 416; by way of non-limiting example, where the computer 404 detects a change in the compensated streaming current value that indicates an increase in the turbidity of the source water, the computer 404 may command the coagulant dosing device 430 to increase the coagulant dosing rate, and where the computer 404 detects a change in the compensated streaming current value that indicates a decrease in the turbidity of the source water, the computer 404 may command the coagulant dosing device 430 to decrease the coagulant dosing rate. In many embodiments, the PID control loop controlled by the computer 404 may be effective not only to maintain a turbidity setpoint but to regulate the speed at which changes in incoming source water are compensated for (e.g., to command faster or slower increases or decreases in the rate at which the coagulant dosing device 430 speeds up or slows down).

In embodiments of any of the automated chemical dosing methods and systems disclosed herein, it is to be expressly understood that a processor, operating system, control loop, etc. may display or report information relating to the streaming content (raw and/or compensated/adjusted) of the source water stream and/or operation of any of the methods and systems disclosed herein in a graphical user interface of a computer, to allow a human operator or technician to verify proper functioning, correct error conditions, etc. This display/report functionality may be implemented by any suitable control system architecture, such as, by way of non-limiting example, supervisory control and data acquisition (SCADA) architectures and the like.

The concepts illustratively disclosed herein suitably may be practiced in the absence of any element which is not specifically disclosed herein. It is apparent to those skilled in the art, however, that many changes, variations, modifications, other uses, and applications of the disclosure are possible, and changes, variations, modifications, other uses, and applications which do not depart from the spirit and scope of the disclosure are deemed to be covered by the disclosure.

The foregoing discussion has been presented for purposes of illustration and description. The foregoing is not intended to limit the disclosure to the form or forms disclosed herein. In the foregoing Detailed Description, for example, various features are grouped together in one or more embodiments for the purpose of streamlining the disclosure. The features of the embodiments may be combined in alternate embodiments other than those discussed above. This method of disclosure is not to be interpreted as reflecting an intention that the claims require more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate embodiment.

Moreover, though the present disclosure has included description of one or more embodiments and certain variations and modifications, other variations, combinations, and modifications are within the scope of the disclosure, e.g., as may be within the skill and knowledge of those in the art, after understanding the present disclosure. It is intended to obtain rights which include alternative embodiments to the extent permitted, including alternate, interchangeable, and/or equivalent structures, functions, ranges, or steps to those claimed, regardless of whether such alternate, interchangeable, and/or equivalent structures, functions, ranges, or steps are disclosed herein, and without intending to publicly dedicate any patentable subject matter.

1. A method for coagulant dosing in a water treatment process, comprising:
(a) collecting data relating to a combined content of insoluble and soluble ions in a source water stream at or upstream of a coagulant dosing device;
(b) collecting data relating to a content of soluble ions in the source water stream downstream of the coagulant dosing device;
(c) calculating, based on the data collected in steps (a) and (b), a soluble ion content-compensated turbidity of the source water stream at or upstream of the coagulant dosing device; and
(d) commanding the coagulant dosing device to increase, decrease, or maintain a coagulant dosing rate to achieve a turbidity setpoint in the source water stream downstream of the coagulant dosing device.

2. The method of claim 1, wherein step (a) is carried out by a streaming current sensor.

3. The method of claim 1, wherein step (a) is carried out in a rapid mixing vessel of the water treatment system.

4. The method of claim 1, wherein step (b) is carried out by a streaming current sensor.

5. The method of claim 1, wherein step (b) is carried out by one or more sensors selected from the group consisting of an electrical conductivity analyzer, a total dissolved solids analyzer, an alkalinity analyzer, a UV254 analyzer, and combinations thereof.

6. The method of claim 1, wherein step (b) is carried out in a clear well of the water treatment system.

7. The method of claim 1, wherein step (c) comprises subtracting a second ion content value measured in step (b) from a first ion content value measured in step (a).

8. The method of claim 1, wherein the data on which the calculation of step (c) is based further comprise source water temperature data, source water pH data, sensor drift data, or a combination thereof.

9. A streaming current monitoring system for a water treatment process, comprising:
a computer;
a first ion content sensor, positioned at or upstream of a coagulant dosing device of the water treatment process and configured to collect data relating to a combined content of insoluble and soluble ions in a source water stream; and
a second ion content sensor, positioned downstream of the coagulant dosing device and configured to collect data relating to a content of soluble ions in the source water stream,
wherein the computer comprises a processor and a non-transitory computer-readable medium storing instructions that, when executed by the processor, cause the processor to perform the steps of:
(a) calculating, based on the data collected by the first and second ion content sensors, a soluble ion content-compensated turbidity of the source water stream at or upstream of the coagulant dosing device; and
(b) at least one of:
(i) displaying the soluble ion content-compensated turbidity of the source water stream in a graphical user interface of the computer; and
(ii) communicating the soluble ion content-compensated turbidity of the source water stream to a controller that controls the coagulant dosing device.

10. The streaming current monitoring system of claim 9, wherein the first ion content sensor is a streaming current sensor.

11. The streaming current monitoring system of claim 9, wherein the first ion content sensor is positioned within a rapid mixing vessel of the water treatment process.

12. The streaming current monitoring system of claim 9, wherein the second ion content sensor is a streaming current sensor.

13. The streaming current monitoring system of claim 9, wherein the second ion content sensor is selected from the group consisting of an electrical conductivity analyzer, a total dissolved solids analyzer, an alkalinity analyzer, a UV254 analyzer, and combinations thereof.

14. The streaming current monitoring system of claim 9, wherein the second ion content sensor is positioned within a clear well of the water treatment process.

15. The streaming current monitoring system of claim 9, wherein step (a) comprises subtracting a second ion content value measured by the second ion content sensor from a first ion content value measured by the first ion content sensor.

16. An automated coagulant dosing system for a water treatment process, comprising:
a computer;
a coagulant dosing device;
a first ion content sensor, positioned at or upstream of the coagulant dosing device and configured to collect data relating to a combined content of insoluble and soluble ions in a source water stream; and
a second ion content sensor, positioned downstream of the coagulant dosing device and configured to collect data relating to a content of soluble ions in the source water stream,
wherein the computer comprises a processor and a non-transitory computer-readable medium storing instructions that, when executed by the processor, cause the processor to perform the steps of:
(a) calculating, based on the data collected by the first and second ion content sensors, a soluble ion content-compensated turbidity of the source water stream at or upstream of the coagulant dosing device; and
(b) commanding the coagulant dosing device to increase, decrease, or maintain a coagulant dosing rate to achieve a turbidity setpoint in the source water stream downstream of the coagulant dosing device.

17. The automated coagulant dosing system of claim 16, wherein the first ion content sensor is a streaming current sensor.

18. The automated coagulant dosing system of claim 16, wherein the first ion content sensor is positioned within a rapid mixing vessel of the water treatment process.

19. The automated coagulant dosing system of claim 16, wherein the second ion content sensor is a streaming current sensor.

20. The automated coagulant dosing system of claim 16, wherein the second ion content sensor is selected from the group consisting of an electrical conductivity analyzer, a total dissolved solids analyzer, an alkalinity analyzer, a UV254 analyzer, and combinations thereof.

21. The automated chemical dosing system of claim 16, wherein the second ion content sensor is positioned within a clear well of the water treatment process.

22. The automated chemical dosing system of claim 16, wherein step (a) comprises subtracting a second ion content value measured by the second ion content sensor from a first ion content value measured by the first ion content sensor.

23. The automated chemical dosing system of claim 16, further comprising at least one of a temperature sensor and a pH sensor.

24. A streaming current sensor calibration system, comprising:
a sensor chamber, surrounding and defining a sensor column and configured to receive and securely hold a streaming current sensor within the sensor column;
a first container enclosing a first interior volume, wherein a first streaming potential standard fluid is contained within the first interior volume; and
one or more calibration fluid dispensers, configured to dispense the first streaming potential standard fluid and a second streaming potential fluid onto a surface of the streaming current sensor to calibrate the streaming current sensor.

25. The streaming current sensor calibration system of claim 24, further comprising a second container enclosing a second interior volume, wherein the second streaming potential standard fluid is contained within the second interior volume.

26. The streaming current sensor calibration system of claim 24, further comprising a sample line in fluid communication with both a stream or vessel containing the second streaming potential standard fluid and the one or more calibration fluid dispensers and configured to convey the second streaming potential standard fluid from the stream or vessel containing the second streaming potential standard fluid to the one or more calibration fluid dispensers.

27. The streaming current sensor calibration system of claim 24, wherein the one or more calibration fluid dispensers consist of a single calibration fluid dispenser.

28. The streaming current sensor calibration system of claim 24, wherein the one or more calibration fluid dispensers comprise a first calibration fluid dispenser, configured to dispense the first streaming potential standard fluid onto the surface of the streaming current sensor, and a second calibration fluid dispenser, configured to dispense the second streaming potential standard fluid onto the surface of the streaming current sensor.

29. The streaming current sensor calibration system of claim 24, wherein the first streaming potential standard fluid comprises a suspension of latex beads in water.

30. The streaming current sensor calibration system of claim 24, wherein the second streaming potential standard fluid consists essentially of filtered or deionized water.
